(19) United States
(12) Patent Application Publication
(10) Pub. No.: US 2021/0290214 A1
(43) Pub. Date: Sep. 23, 2021

(54) MEDICAL DEVICES FOR SHUNTS, OCCLUDERS, FENESTRATIONS AND RELATED SYSTEMS AND METHODS

(71) Applicant: W. L. Gore & Associates, Inc., Newark, DE (US)

(72) Inventors: Daniel S. Cole, Flagstaff, AZ (US); Tom R. McDaniel, Flagstaff, AZ (US); Edward E. Shaw, Flagstaff, AZ (US); Benjamin A. Smith, Flagstaff, AZ (US)

(21) Appl. No.: 17/260,167

(22) PCT Filed: Jul. 17, 2019
(86) PCT No.: PCT/US2019/042252

§ 371 Date: Jan. 13, 2021

Related U.S. Application Data
(60) Provisional application No. 62/699,815, filed on Jul. 18, 2018.

(51) Int. Cl. A61B 17/00; A61B 17/11; A61B 5/00

(52) U.S. Cl. CPC A61B 17/0057; A61B 17/11; A61B 5/686; A61B 2562/0247; A61B 2017/00597; A61B 2017/1139; A61B 2017/00592

(57) ABSTRACT

An implantable medical device comprising a first frame component. The first frame component including a first set of elongate elements configured to conform to an anatomy of a patient. The implantable medical device also comprising a second frame component including a second set of elongate elements configured to conform to an anatomy of a patient. The first frame component and the second frame component being discrete and separate from one another. The implantable medical device also comprising a conduit portion arranged between the first frame component and the second frame component. The conduit portion including a membrane connecting the first frame component and the second frame component.

### CROSS-REFERENCE TO RELATED APPLICATION

This application is a national phase application of PCT Application No. PCT/US2019/042252, internationally filed on Jul. 17, 2019, which claims the benefit of Provisional Application No. 62/699,815, filed Jul. 18, 2018, both of which are incorporated herein by reference in their entireties for all purposes.

### FIELD

The present disclosure relates generally to implantable medical devices, and more specifically to implantable medical devices for shunting and/or occluding bodily fluids or structures and related systems and methods thereof.

### BACKGROUND

Heart failure and diseases of the heart affect millions of people worldwide. Heart failure includes failure of either the left side of the heart, the right side of the heart, or both. Diseases of the heart that can lead to heart failure include hypertension, pulmonary arterial hypertension, and congenital defects of the heart. The constantly evolving nature of heart failure represents a significant challenge for the treatment methods. Therefore, there is a need for new and adaptable methods and devices for treating heart failure.

### SUMMARY

In one example (“Example 1”), an implantable medical device includes a first frame component configured to conform to an anatomy of a patient; a second frame component configured to conform to an anatomy of a patient wherein the first frame component and the second frame component are discrete and separate from one another; and a conduit portion arranged between the first frame component and the second frame component, the conduit portion including a membrane connecting the first frame component and the second frame component.

In another example (“Example 2”), further to the device of Example 1, where at least a portion of the conduit portion is radially unsupported by the first and second frame components within the conduit portion.

In another example (“Example 3”), further to the device of Example 2, the first and second frame components are configured to facilitate deployment of the conduit portion and maintaining a lumen through the conduit portion.

In another Example (“Example 4”), further to the device of any one of Examples 1-3, the conduit portion is free of frame components.

In another Example (“Example 5”), further to the device of any one of Examples 1-4, the first frame component includes a first set of elongate elements and the second frame component includes a second set of elongate elements, and the first set of elongate elements and the second set of elongate elements are non-contiguous with one another.

In another Example (“Example 6”), further to the device of Example 5, the first set of elongate elements include a first plurality of support struts and wherein the second set of elongate elements include a second plurality of support struts, the first and second plurality of support struts forming a support structure within each of the elongate elements.

In another example (“Example 7”), further to the device of Example 6, the first set of elongate elements form a plurality of first lobes.

In another example (“Example 8”), further to the device of Example 6, the first set of elongate elements form a star shape.

In another Example (“Example 9”), further to the device of any one of Examples 5-8, the first frame component forms a first side and the second frame component forms a second side, and wherein at least one of the first set of elongate elements are arranged within the first frame component without crossing into the second side and the second set of elongate elements are arranged within the second frame component without crossing into the first side.

In another Example (“Example 10”), further to the device of Example 9, at least one of the first set of elongate elements and the second set of elongate elements extend within the conduit portion.

In another Example (“Example 11”), further to the device of any one of Examples 1-10, the membrane extends to at least partially cover portions of one or both of the first frame component and the second frame component.

In another Example (“Example 12”), further to the device of Example 11, the membrane is configured to promote tissue ingrowth to cover at least a portion of one or both of the first frame components and second frame components.

In another Example (“Example 13”), further to the device of any one of Examples 1-12, the device also includes a first membrane film arranged on first frame component and a second membrane film arranged on the second frame component.

In another Example (“Example 14”), further to the device of any one of Examples 1-3, the membrane separates the first frame components and second frame components by a gap of from 0 to 15 mm.

In one example (“Example 15”), an implantable medical device for regulating blood pressure between a left and right atrium of a heart includes: a conduit portion configured to span a septum of the heart and configured to allow fluid flow therethrough; and a frame component including a first set of elongate elements arranged on a first side of the conduit portion and a second set of elongate elements arranged on a second side of the conduit portion with the first set of elongate elements and the second set of elongate elements being non-contiguous with one another.

In another example (“Example 16”), further to the device of Example 15, the frame component forms a first side including the first set of elongate elements and a second side including the second set of elongate elements, and wherein the first set of elongate elements are arranged within the first side and the conduit portion and the second set of elongate elements are arranged within the first side and the conduit portion.

In another Example (“Example 17”), further to the device of any one of Examples 15-16, the first set of elongate members and the second set of elongate members extend radially outward from the conduit portion to form first and second angles, and wherein the first and second angles are approximately 90° angles with respect to the conduit portion.

In another Example (“Example 18”), further to the device of any one of Examples 15-17, the device also includes a sensor arranged with the conduit portion or the frame component and configured to sense at least one of physiologic properties, hemodynamics, biomarkers, sound, pressure, and electrolytes.

In another Example (“Example 19”), further to the device of any one of Examples 15-18, the device also includes at least one of a coating of heparin to facilitate thromboresistance and patency of the device and a coating of paclitaxel to modulate tissue/cellular response.

In one Example (“Example 20”), a method for regulating blood pressure between a left and right atrium of a heart includes delivering the implantable medical device to a desired treatment location within a body of a patient, the implantable medical device comprising: a conduit portion configured to span a septum of the heart and configured to allow fluid flow therethrough; a frame component including a first set of elongate elements arranged on a first side of the conduit portion and a second set of elongate elements arranged on a second side of the conduit portion with the first set of elongate elements and the second set of elongate elements being non-contiguous with one another; positioning the device such that the conduit portion spans a septum between the left and right atrium of the heart; and deploying the first frame component and the second frame component such that the conduit portion opens a desired amount to provide a fluid flow path between the left and right atrium.

In another Example (“Example 21”), further to the method of Example 20, the method also includes adjusting tension on the device to adjust a diameter of the conduit portion and a fluid flow velocity therethrough.

According to one Example (“Example 22”) an implantable medical device includes a first frame component; a second frame component, wherein the first frame component and the second frame component are discrete and separate from one another; and a conduit portion arranged between the first frame component and the second frame component including a membrane connecting the first frame component and the second frame component configured to expand in response tension in the conduit portion imparted by expansion of the first and second frame components.

In another Example (“Example 23”), further to the device of Example 22, the conduit portion is configured to span a septum between left and right atrium of a patient's and the conduit portion is configured to expand the septum in response tension in the conduit portion imparted by expansion of the first and second frame components.

In another Example (“Example 24”), further to the device of Example 23, the conduit portion is configured to maintain an expanded diameter of the septum.

In another Example (“Example 25”), further to the device of Example 22, the first frame component forms a first side and the second frame component forms a second side, and wherein at least one of the first set of elongate elements are arranged within the first frame component without crossing into the second side and the second set of elongate elements are arranged within the second frame component without crossing into the first side.

In another Example (“Example 26”), further to the device of Example 25, at least one of the first set of elongate elements and the second set of elongate elements extend within the conduit portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example implantable medical device for regulating blood pressure in accordance with an embodiment.

FIG. 2 is an example implantable medical device for regulating blood pressure in accordance with an embodiment.

FIG. 3A is a perspective view of another example implantable medical device for regulating blood pressure in accordance with an embodiment.

FIG. 3B is a side view of the implantable medical device for regulating blood pressure, shown in FIG. 3A, in accordance with an embodiment.

FIG. 4 is an example implantable medical device in accordance with an embodiment.

FIG. 5A is a first perspective view of another example implantable medical device for regulating blood pressure in accordance with an embodiment.

FIG. 5B is a second perspective view of the implantable medical device for regulating blood pressure, shown in FIG. 5A, in accordance with an embodiment.

FIG. 5C is a third perspective view of an implantable medical device for regulating blood pressure, in accordance with an embodiment.

FIG. 6 is an example stent-pattern for an implantable medical device for regulating blood pressure and deployment system in accordance with an embodiment.

FIG. 7 is another example stent-pattern for an implantable medical device for regulating blood pressure and deployment system in accordance with an em bodim ent.

FIGS. 8A-8B show an example implantable medical device arranged on a mandrel in accordance with an embodiment.

FIGS. 8C-8D show the example implantable medical device of FIGS. 8A and 8B implanted within the body of a patient in accordance with an embodiment.

FIG. 9 is another example implantable medical device in accordance with an embodiment.

FIG. 10 is another example implantable medical device in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward to implantable medical devices such as device for shunting and/or occluding bodily fluids or structures. In certain instances, the various aspects of the present disclosure relate to methods and devices for treating heart failure by reducing elevated blood pressure in a heart chamber by creating a pressure relief shunt. Additionally, some embodiments relate to methods and devices for customizing, adjusting or manipulating the flow of blood through the shunt in order to enhance the therapeutic effect of the pressure relief shunt.

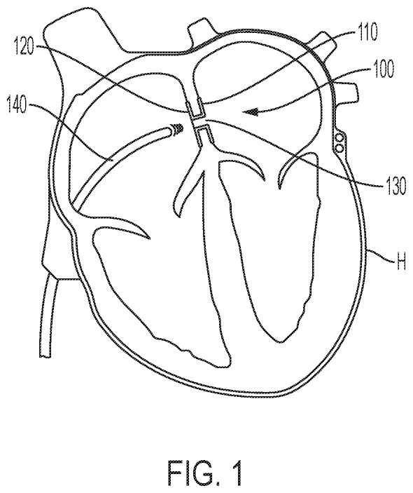

FIG. 1 is an example implantable medical device for regulating blood pressure in accordance with an embodiment. The implantable medical device 100 is shown implanted within a heart H of a patient. The device 100 is shown arranged between the patient's left atrium and right atrium. In certain instances, the device 100 may be used to regulate blood flow within the heart H, for example, between the left and right atriums LA, RA. As shown, the device 100 generally includes a first frame component 110 arranged on a first side of a septum (e.g., within the right atrium RA), a second frame component 120 arranged on a second side of the septum (e.g., within the left atrium LA), and a conduit portion 130 extending through the septum. A needle may be used to create an opening in the septum.

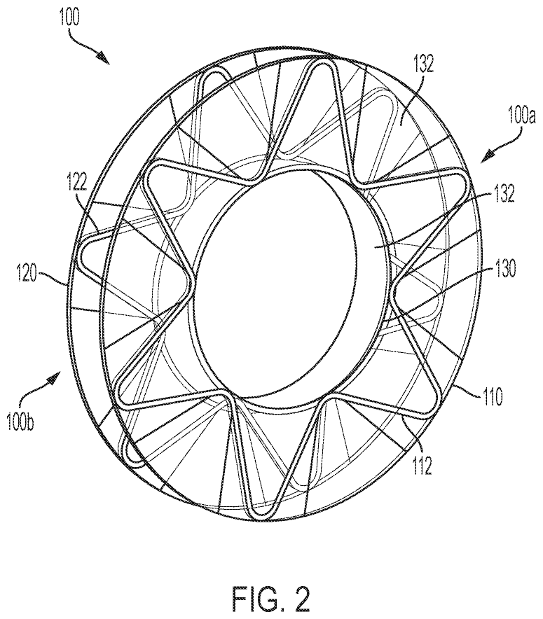

A sheath 140 and constraining and/or release lines (not shown) may be used to facilitate deployment of the device 100. For example, a first side of the device 100 that includes the first frame component 110 may be released after the sheath 140 is advanced through the septum and to the RA, and the second side 120 that includes the second frame component 120 may be released on the LA side of the septum. A conduit portion 130 (e.g., shown in FIG. 2) is arranged within the opening. The frame components 110, 120 and the conduit portion 130 may be compressed within the sheath 140 during delivery of the device 100 to the desired treatment area within the patient and subsequently expanded during deployment of the device 100.

FIG. 2 is an example implantable medical device for regulating blood pressure in accordance with an embodiment. As shown, the device 100 includes the first frame component 110 and the second frame component 120. The first frame component 110 may be configured to conform to the patient's anatomy (i.e., the first side of the septum, for example). The second frame component 120 may be configured to conform to the patient's anatomy (i.e., the second side of the septum).

In certain instances, the first frame component 110 includes a first set of elongate elements 112, and the second frame component 120 includes a second set of elongate elements 122. The frame components 110, 120, including and for example the elongate elements 112, 122, may be discrete and separate from one another. For example, the first frame component 110 forms a first side 100a of the device 100 and the second frame component 120 forms a second side 100b of the device 100. The first frame component 110 being discrete and separate from the second frame component 120 does not enter into the second side 100b of the device and the second frame component 120 being discrete and separate from the first frame component 110 does not enter into the first side 100a of the device.

In certain instances, the first and second frame components 110, 120 are non-contiguous with one another. The first and second frame components 110, 120 being non-contiguous with one another allows the first and second frame components 110, 120 to be distinct and separate from one another. In addition, the first and second frame components 110, 120 are free to move, in response to movement of the patient's anatomy, separate from one another. In this manner, forces acting on one of the first and second frame components 110, 120 are maintained within the other of the first and second frame components 110, 120. The forces acting on one of the first and second frame components 110, 120 may be isolated to the frame component to which the force is acted on.

As shown, the conduit portion 130 is arranged between the first frame component and the second frame component. At least a portion of the conduit portion 130 is generally radially or circumferentially unsupported by the first and second frame components 110, 120 within the conduit portion 130. As shown in FIG. 2, the conduit portion 130 transitions to the first side 100a and the second side 100b at approximately a 90 degree angle (other angles are contemplated). Bounds of the conduit portion 130 may be considered to be a location at which the conduit portion 130 transitions to the first side 100a and the second side 100b. The first and second frame components 110, 120 extend laterally relative to the conduit portion 130. In addition, the first and second frame components 110, 120 may support the conduit portion 130 without substantially entering the bounds of the conduit portion 130. In certain instances, the first and second frame components 110, 120 support the conduit portion 130 laterally from outside of bounds the conduit portion 130. Thus, the first and second frame components 110, 120 may maintain a lumen through the conduit portion 130 and facilitate deployment of the conduit portion 130 by laterally forcing the conduit portion 130 open.

In certain instances, the first and second frame components 110, 120 may impart tension to the conduit portion 130 to deploy and maintain the conduit portion 130 with a lumen therethrough. The conduit portion 130 may be deployed within the septum between tissue surfaces through an opening (e.g., needle stick across the septum) that has a diameter smaller than a fully deployed diameter of the conduit portion 130. Tension in the conduit portion 130 imparted by expansion of the first and second frame components 110, 120 may also expand the septum between tissue surfaces to a desired shunt size.

In certain instances, the conduit portion 130 may be substantially free of frame components. For example, because the first and second frame components 110, 120 are non-contiguous with one another, as described above, and are arranged external to the bounds of the conduit portion 130. The conduit portion 130 may include, for example, a membrane 132, such as an expanded polytetrafluoroethylene (ePTFE) membrane, connecting the first frame component 110 and the second frame component 120. The membrane 132 generally separates the first frame component 110 and the second frame component 120 by a suitable distance compatible with the patient's body. For example, the membrane 132 can separate the first frame component 110 and the second frame component 120 by a gap of from 0 to 15 mm depending on the desired treatment location within the patient's body. In addition, the conduit portion may be formed of only the membrane 132. The conduit portion 130, which is configured to be deployed within the septum between tissue surfaces, is free of the first frame component 110 and the second frame component 120. The conduit portion 130 may include a smooth interior that facilitates blood flow therethrough without ridges from a stent element interrupting or disrupting flow. Thus, the conduit portion 130 may lessen the opportunity for thrombosis.

In addition to the membrane 132 forming the conduit portion 130, the membrane 132 may also cover at least a portion of the first frame component 110, at least a portion of the second frame component 120, or at least a portion of the first frame component 110 and the second frame component 120. In certain instances, the membrane 132 arranged on at least a portion of the first frame component 110 and/or the second frame component 120 is a separate membrane film (e.g., a first membrane film arranged on first frame component 110 and a second membrane film arranged on the second frame component 120). In these instances, the membrane film or films may be coupled to the membrane 132 in the conduit portion 130. The membrane 132 may be elastic to allow for expansion of the conduit portion 130 and to allow for movement of portions of the first frame component 110 and/or the second frame component 120 (e.g., movement of the first set of elongate elements 112 and/or the second set of elongate elements 122).

The membrane 132 may span gaps between the first set of elongate elements 112 and/or the second set of elongate elements 122. The membrane 132, in certain instances, is arranged on at least a tissue engaging side of the first frame component 110 and a tissue engaging side the second frame component 120. In these instances, the membrane 132 is configured to lessens frame erosion potential of the first frame component 110 and/or the second frame component 120. The membrane 132 and the arrangement of the first set of elongate elements 112 and/or the second set of elongate elements 122 may conform to the tissue surfaces surrounding the septum. The first set of elongate elements 112 and/or the second set of elongate elements 122 may lay flat against the tissue surfaces.

In certain instances, each of the first set of elongate elements 112 may be attached to one another via the membrane 132 to form the first frame component 110. In certain instances, the first frame component 110 may form a substantially flat or 2-dimensional, disc-like shape, as shown. Additionally, or alternatively, the second set of elongate elements 122 may also be attached to one another via the membrane material 132 to form the second frame component 120. The second frame component 120 may also form a substantially flat or 2-dimensional, disc-like shape such that the first and second frame components 110, 120 are substantially parallel to one another when the device 100 is in a deployed configuration.

In certain instances, the membrane 132 may be configured to promote tissue ingrowth over at least a portion of the membrane 132, or at least a portion of the membrane 132. In certain instances, the membrane 132 is configured to promote tissue ingrowth to cover at least a portion of the first and/or second frame components 110, 120, which may further promote compatibility and stability of the device 100 within the patient's body. The membrane 132 within the conduit portion 130 may be configured to not allow tissue ingrowth leading to increased patency. In certain instances, the membrane 132 is configured to promote endothelization without obstructive ingrowth within the conduit portion 130. The membrane 132 may promote endothelization without obstructive overgrowth of tissue into the conduit portion 130.

In certain instances, the device 100 may be capable of delivering a drug to the desired treatment location within the patient's body. For example, the device 100 may be capable of eluting a drug configured to modulate tissue response. In certain instances, the device 100 may be coated with a therapeutic coating, drug eluting material or other therapeutic material or a hydrophilic coating. In one specific example, the device 100 can be coated with heparin to facilitate thromboresistance and patency of the device 100. Alternatively, or additionally, the device 100 may include paclitaxel (to modulate tissue/cellular response).

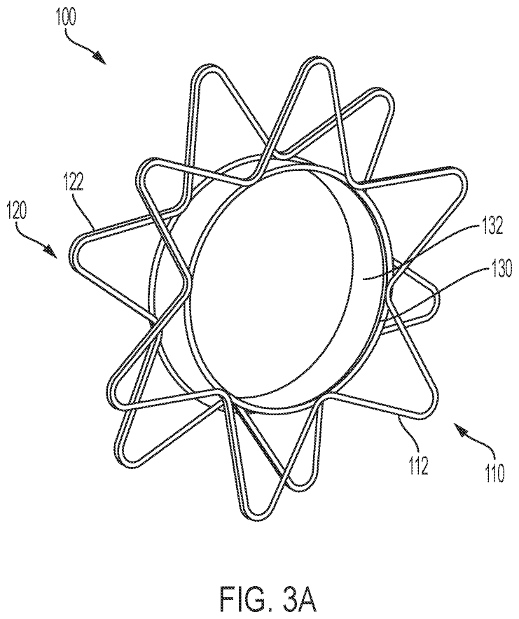

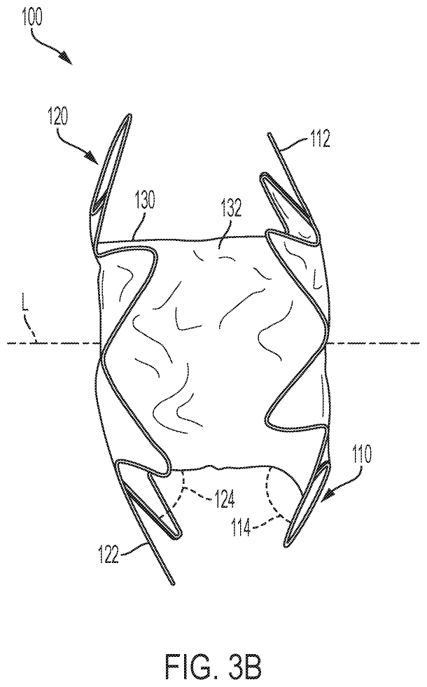

FIG. 3A is a perspective view of another example implantable medical device 100 for regulating blood pressure in accordance with an embodiment. As shown, each of the first set of elongate elements 112 may be discrete and separate from adjacent elongate elements. In other terms, the membrane 132 does not connect each of the first set of elongate elements 112 together. In this way, each of the first set of elongate elements 112 may move independently from one another and individually conform to the topography of the first side of the septum, thus providing a highly conformable first frame component 110. Each of the second set of elongate elements 122 may also be discrete and separate from adjacent elongate elements. For example, each of the second set of elongate elements 122 may move independently from one another and individually conform to the second side of the septum, much like the first set of elongate elements 112 conforms to the first side of the septum. Thus, both the first and second frame components 110, 120 are highly conformable and may conform independently of one another based on the patient's anatomy.

In certain instances, one of the first or second set of elongate elements 112, 122 of the first and second frame components 110, 120 may be attached to one another via the membrane 132 while the other set of elongate elements are unattached (e.g., they are discrete and separate from adjacent elongate elements). In other instances, only some of the first or second set of elongate elements 112, 122 may be attached to one another, while other elongate elements of the first and second set of elongate elements 112, 122 are not attached. Thus, the device 100 can be highly customizable to the patient depending on the desired treatment location within the patient, and size and/or shape of the defect, among other factors.

The device 100 is generally deployable or expandable from a delivery configuration to the deployed configuration. In some instances, the first set of elongate elements 112 and the second set of elongate elements 122 may nest within one another when the device is in the delivery configuration. This allows the device 100 to compress to a smaller size, for example, for delivery of the device 100 to a wider variety of treatment locations (e.g., through small, narrow, or convoluted passageways).

FIG. 3B is a side view of the implantable medical device for regulating blood pressure, shown in FIG. 3A, in accordance with an embodiment. FIG. 3B shows the device 100 in the deployed configuration. As shown, the first frame component 110 including the first set of elongate elements 112 and the second frame component 120 including the second set of elongate elements 122 are positioned radially outward with respect to a longitudinal axis L of the conduit portion 130 when the device 100 is in the deployed configuration. For example, the first and second frame components 110, 120 are positioned at first and second angles 114, 124, respectively. The first and second angles 114, 124 may form approximately a 90° angle with respect to the longitudinal axis L when the device is in the deployed configuration. This allows the first and second frame components 110, 120 to be positioned parallel with and adjacent to the first and second sides of the septum. In certain instances, the first and second frame components 110, 120 may be positioned at any angle relative to the longitudinal axis L (for example, from about 0° to greater than 90° with respect to the longitudinal axis L) that allows for contact with the tissue surface of the first and second sides of the septum.

In certain instances, the first and second elongate elements 112, 122 are configured to separate from one another when the device 100 is in the deployed configuration. As shown in FIG. 3B, each of the first set of elongate elements 112 are discrete and separate from one another when the device 100 is in the deployed configuration such that each of the first set of elongate elements 112 may move independently from adjacent elongate elements. Each of the second set of elongate elements 122 may also be discrete and separate from one another when the device 100 is in the deployed configuration such that each of the second set of elongate elements 122 move independently from adjacent elongate elements.

The first and second frame components 110, 120 may maintain a lumen through the conduit portion 130 and facilitate deployment of the conduit portion 130 by laterally forcing the conduit portion 130 open. In addition, the lumen may be free or without the first and second frame components 110, 120. In this manner, the conduit portion 130 may facilitate re-crossing of the septum for addition procedures (e.g., left atrial appendage occluder implantation). In addition, the first and second frame components 110, 120 may be differently configured. For example, one of the first and second frame components 110, 120 may be flared while the other of the first and second frame components 110, 120 is flat. In other instances, both the first and second frame components 110, 120 may be flared. In addition, one of the first and second frame components 110, 120 may be convex while the other of the first and second frame components 110, 120 is flat or concave or both the first and second frame components 110, 120 may be convex. Further, one of the first and second frame components 110, 120 may be concave while the other of the first and second frame components 110, 120 is flat or convex or both the first and second frame components 110, 120 may be concave. In addition, the first and second frame components 110, 120 may be different sizes.

The first and second frame components 110, 120 may include a sensor integrated into the respective frame component, for example, for continuous monitoring of various hemodynamic parameters such as pressure, among other parameters, within the patient's body. For example, an antenna or inductor may be wrapped around the perimeter of one of the first and second frame components 110, 120 and the sensor may be attached to the inductor. The sensor may be configured to, for example, sense physiologic properties, such as temperature, electrical signals of the heart, blood chemistry, blood pH level, hemodynamics, biomarkers, sound, pressure, and electrolytes that may be important in diagnosing, monitoring, and/or treating heart disease, heart failure, and/or other cardiovascular disease states

In certain instances, the conduit portion 130 may be sizeable after delivery. The membrane 132 may be selectively adjustable by a balloon applied within the conduit portion 130 to distend the membrane 132. The device 100 can be any size suitable to fit the anatomy of the patient. In certain instances, a diameter of the conduit portion is from 3 to 12 mm. For example, the diameter of the conduit portion may be from 4 to 10 mm, or from 5 to 8 mm depending on the anatomy of the patient and/or the desired treatment location. The first and second frame components 110, 120 generally have a larger diameter than that of the conduit portion 130, for example, so that the frame components may anchor the conduit portion 130 of the device 100 within the septum.

The device 100 can be any shape suitable to fit the anatomy of the patient. For example, the first and second frame portions 110, 120 may be any of a variety of suitable shapes for anchoring the device 100 within the patient's body. For example, the first and second frame portions 110, 120 may be substantially circular, ovular, diamond-shaped, star-shaped, flower-shaped, or any other suitable shape as desired. In certain instances, for example, at least one of the first and second set of elongate elements 112, 122 form a star shape. In certain instances, both the first and second set of elongate elements 112, 122 form a star shape.

In certain instances, the first set of elongate elements 112 forms a plurality of first lobes 116 and the second set of elongate elements 122 forms a plurality of second lobes 126. Each of the plurality of first and second lobes 116, 126 may include, for example, from 3 to 12 lobes, from 4 to 10 lobes, or from 6 to 8 lobes as desired. In certain instances, the plurality of first lobes 116 may have more lobes than the plurality of second lobes 126, while in other instances, the plurality of first lobes 116 may have the same number of lobes or less lobes than the plurality of second lobes 126.

FIG. 4 is another example implantable medical device 100 in accordance with an embodiment. In certain instances, the device 100 may include a cover 160 positioned over at least a portion of the conduit portion 130. The cover 160 can be formed, for example, by a graft material such as the material used for the membrane 132. The cover 160 is configured to reduce the amount of fluid passing through the conduit portion 130 or, in certain instances, to prevent fluid from passing through the conduit portion 130 altogether. Thus, the cover 160 may partially or fully occlude the septum as desired. The device 100 may be an occluder in these instances. When used as an occluder, the expanded conduit portion 130 may center with in the target location (e.g., within the defect).

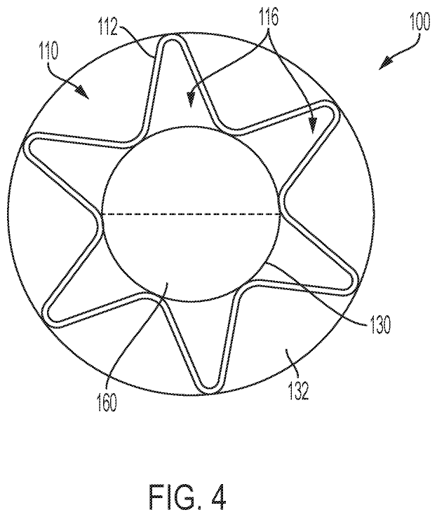

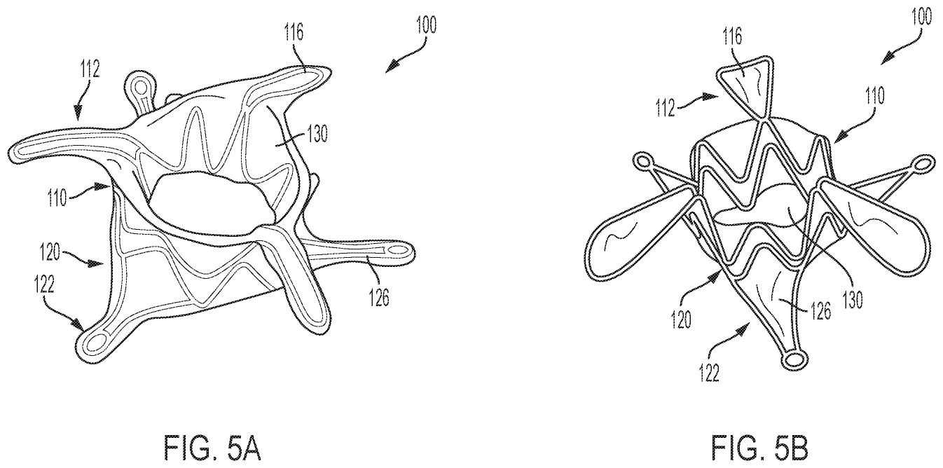

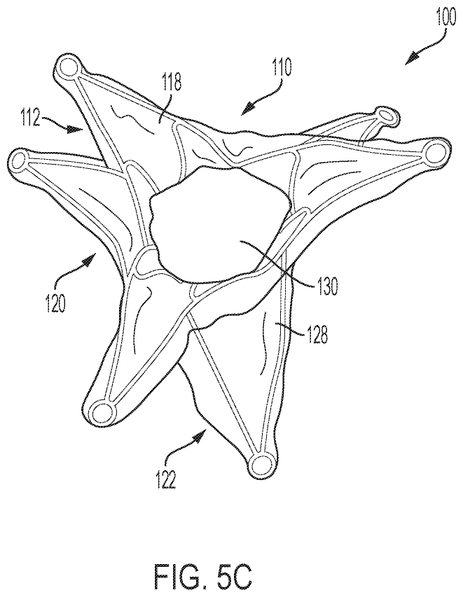

FIGS. 5A-5C show a perspective views of other example implantable medical devices for regulating blood pressure in accordance with various embodiments. As shown, the plurality of first and second lobes 116, 126 of the first and second set of elongate elements 112, 122 may have a variety of shapes. For example, each lobe of the first and second set of elongate elements 112, 122 may be generally elongate, triangular, rhomboid, or petal-like in shape.

FIG. 5A is a first perspective view of another example implantable medical device for regulating blood pressure in accordance with an embodiment. As shown, the first and second set of elongate elements 112, 122 form the first and second frame portions 110, 120 of the device 100, and each of the first and second set of elongate elements 112, 122 include three lobes. As shown, the plurality of first lobes 116 and/or the plurality of second lobes 126 can be somewhat elongate in shape.

The device 100 is generally deployable or expandable from the delivery configuration to the deployed configuration. In some instances, the first set of elongate elements 112 and the second set of elongate elements 122 nest within one another when the device is in the delivery configuration. This allows the device 100 to compress to a smaller size, for example, for delivery of the device 100 to a wider variety of treatment locations (e.g., through small, narrow, or convoluted passageways).

As shown in FIG. 5B, the first set of elongate elements 112 may have a generally different shape than the second set of elongate elements 122. For example, the plurality of first lobes 116 is elongate in shape, while the plurality of second lobes 126 is triangular in shape. As shown, the first set of elongate elements 112 is attached to the first frame component 110 and the second set of elongate elements 122 is attached to the second frame component 120. Both of the first and second frame components 110, 120 may extend into the conduit portion 130. However, neither of the first and second frame components 110, 120 extend into the opposite frame component (e.g., the first frame component 110 does not extend into the second side 100b and the second frame component 120 does not extend into the first side 100a). In certain instances, the stent or frame elements arranged within the conduit portion 130 may be formed of a third frame component as is described in further detail with reference to FIG. 10. The first and second frame components 110, 120 may maintain a lumen through the conduit portion 130 and facilitate deployment of the conduit portion 130 by laterally forcing the conduit portion 130 open. FIG. 5C is a third perspective view of the implantable medical device for regulating blood pressure, in accordance with an embodiment. As shown, in certain instances, the first set of elongate elements 112 may include a first plurality of support struts 118 and the second set of elongate elements 122 may include a second plurality of support struts 128. The first and second plurality of support struts 118, 128 generally form a support structure within each of the elongate elements, which may increase the strength and/or stability of each of the elongate elements. In certain instances, the support struts 118, 128 may also aid in delivery of the device 100. For example, the support structures may provide a location in which a delivery device may be easily attached to the device 100 when the device 100 is in the delivery configuration.

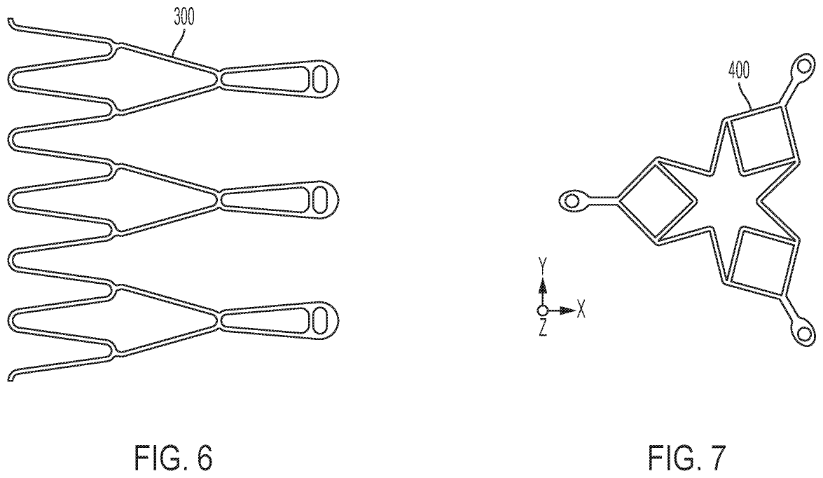

FIG. 6 is an example stent pattern for an implantable medical device for regulating blood pressure in accordance with an embodiment. In certain instances, a stent pattern 300 can be used for the process of creating the device 100. For example, in certain instances, the device 100 is made from a tube or sheet of material, such as Nitinol (NiTi) or stainless steel, that is cut according to the stent pattern 300 and is then expanded to the configuration of the device 100 as shown in FIGS. 2-5.

FIG. 7 is another example stent pattern for an implantable medical device for regulating blood pressure in accordance with an embodiment. As described above, the stent pattern 400 can be used for the process of creating the device 100. For example, in certain instances, the device 100 is made from a tube or sheet of material, such as Nitinol (NiTi) or stainless steel, that is cut according to the stent pattern 300 and is then expanded to the configuration of the device 100 as shown in FIGS. 2-5. In other instances, the stent components may be formed by wires wound around a jig and then heat set.

The device 100 of FIGS. 6 and 7 is generally deployable or expandable from a delivery configuration to the deployed configuration. In some instances, the first set of elongate elements 112 (not shown) and the second set of elongate elements 122 (not shown) may nest within one another when the device is in the delivery configuration. This allows the device 100 to compress to a smaller size, for example, for delivery of the device 100 to a wider variety of treatment locations (e.g., through small, narrow, or convoluted passageways).

In certain instances, the devices 100 as discussed herein may include multiple frame components. The frame components may be formed of separate tubes or sheets of material or from a single tube or sheet of material. in other instances, each of the frame components may be individually formed from one or wires or the frame components may be formed together by one or more wires. Elongate elements, as discussed herein, may be struts, wires, or portions of the tube(s) or sheet(s) of material form portions of the frame components.

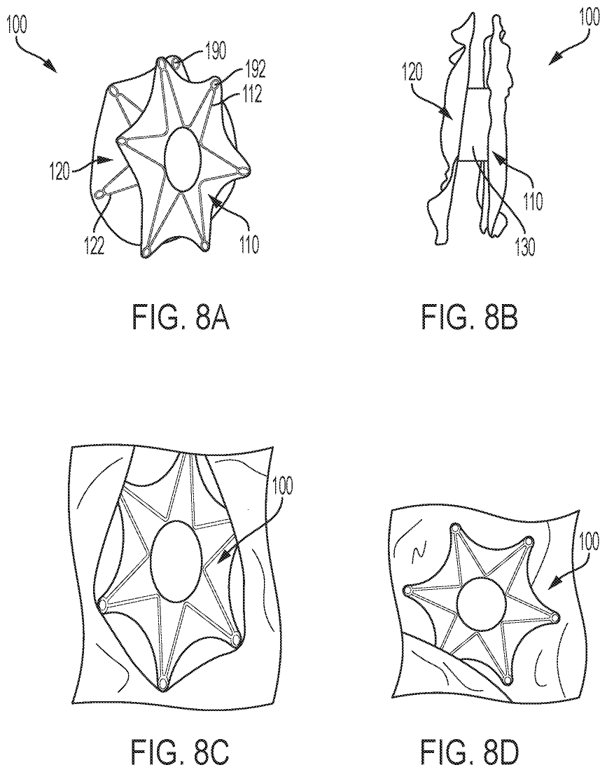

FIGS. 8A-8D show the implantable medical device 100 in accordance with an embodiment. FIGS. 8A and 8B show the device 100 arranged on a mandrel. As shown, the device 100 includes opposing first and second frame portions 110, 120 having first and second series of elongate elements 112, 122 positioned radially outward from the conduit portion 130, as shown in FIGS. 2, 3A, and 3B. Each of the first and second series of elongate elements 112, 122 may include eyelets configured to aid in delivery of the device 100. For example, the first series of elongate elements 112 includes a first plurality of eyelets 190 and the second series of elongate elements 122 includes a second plurality of eyelets 192. FIGS. 8C and 8D show the device 100 of FIGS. 8A and 8B implanted at a desired treatment location within the body of a patient. The eyelets 190, 192 may interface with a wire or suture-like element on a delivery system for constraining of portions of the implantable medical device 100 and, in certain instances, re-capturability of the device 100.

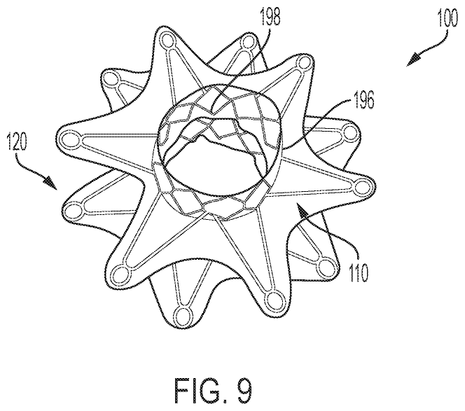

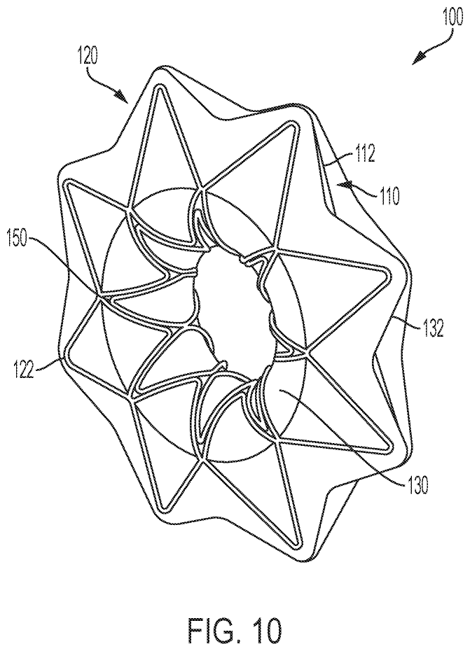

FIG. 9 is another example implantable medical device 100 in accordance with an embodiment. As shown, the first frame portion 110 may include a first protruding portion 196 extending outward along the longitudinal axis from the surface of the first frame portion 110. The second frame portion 120 may also include a second protruding portion 198 extending outward along the longitudinal axis from the surface of the second frame portion 120. The first and second protruding portions 196, 198 can be formed, for example, of a stent frame. The first and second protruding portions 196, 198 may be independent from the first frame portion 110 and the second frame portion 120. The protruding portions 196, 198 can intersect the first frame portion 110 and the second frame portion 120 at 90 degrees (e.g., as shown) or angles greater than or less than 90 degrees such as 30 degrees, 35 degrees, 40 degrees, 45 degrees, 50 degrees, 55 degrees, 60 degrees, 65 degrees, 70 degrees, 75 degrees, 80 degrees, 85 degrees or any angle therebetween. The first and second protruding portions 196, 198 may be sizeable after delivery of the device 100 to the body or the patient.

In certain instances, a method for regulating blood pressure includes delivering the device 100 to the desired treatment location within the patient's body while the device 100 is in the delivery configuration. The device 100 may then be positioned such that the conduit portion 130 spans the septum between the left and right atrium of the heart, for example, or spans any other defect within the patient's body as desired. The device 100 is then expanded from the delivery configuration to the deployed configuration such that first and second frame components 110, 120 extend radially outward from the conduit portion 130 so that the conduit portion 130 opens a desired amount to provide a fluid flow path through the device 100 (e.g., in certain instances, between the left and right atriums). In certain instances, the tension on the device 100 may be adjusted to further adjust the diameter of the conduit portion 130 of the device 100. This may adjust the fluid flow velocity through the device 100, for example, and allow more or less fluid to pass through the conduit portion 130 of the device 100 as desired.

FIG. 10 is another example implantable medical device in accordance with an embodiment. The device 100 may be used for regulating blood pressure in accordance with an embodiment. As shown, the device 100 may include a first frame portion 110 and a second frame portion 112. As described in further detail below, the device 100 includes a third frame component 150 that may be configured to prop open a conduit portion 130 by self-expanding or balloon expansion. As shown in FIG. 10, the device 100 the first frame component 110 may be arranged on a first side of a septum and the second frame component 120 may be arranged on a second side of the septum. Each of the frame portions 110, 120 and the conduit portion 130 may include a membrane 132. The membrane 132 may cover at least a portion of the first frame component 110, at least a portion of the second frame component 120, or at least a portion of the first frame component 110 and the second frame component 120. The membrane 132 may be elastic to allow for expansion of the conduit portion 130 and to allow for movement of portions of the first frame component 110 and/or the second frame component 120.

In certain instances, first and second set of elongate elements 112, 122 (e.g., struts, wires, frame elements, stent elements) form the first and second frame portions 110, 120 of the device 100. As shown in FIG. 10, the first set of elongate elements 112 form the first frame component 110 and the second set of elongate elements 122 form the second frame component 120. Both of the first and second frame components 110, 120 may extend into the conduit portion 130. In certain instances, neither of the first and second frame components 110, 120 extend into the opposite frame component (e.g., the first frame component 110 does not extend into the second side 100b and the second frame component 120 does not extend into the first side 100a).

In addition, the third frame component 150 (e.g., stent or frame elements) may be arranged within the conduit portion 130. The third frame component 150 may be in addition to the first and/or second frame components 110, 120 that extend into the conduit portion 130 or the third frame component 150 may be the sole frame or stent component within the conduit portion 130. The third frame component 150 may be uncoupled or unconnected to either of both of the first and second frame components 110, 120.

In certain instances, the conduit portion 130 may be sizeable after delivery. In certain instances, the third frame component 150 is balloon expandable. After implantation, the conduit portion 130 may be size adjustable by way of the third frame component 150 being configured to expand. The conduit portion 130 may be sized by the balloon to the desired diameter. in certain instances, the third frame component 150 may be configured to self-expand. In addition, the third frame component 150 may be configured to prop open the conduit portion 130 and, in certain instances, prop open the surrounding tissue.

In certain instances, the first and second frame components 110, 120 are self-expanding. The first and second frame components 110, 120 may be configured to conform to the tissue on either side of the septum. In certain instances, the first set of elongate elements 112 may move independently from one another and individually conform to the topography of the first side of the septum and the second set of elongate elements 122 may move independently from one another and individually conform to the second side of the septum. Thus, both the first and second frame components 110, 120 may be highly conformable and may conform independently of one another based on the patient's anatomy.

The devices 100 discussed herein may be removable. In certain instances, the devices 100 may be replaced or removed if treatment is no longer effective or needed. As noted above, the devices 100 may be coated with a drug such as paclitaxel to deliver the drug to the target anatomical location. In certain instances, use of certain pharmaceuticals could prevent healing of a distended septal puncture resulting in the formation of an atrial shunt. The devices 10 may act as a pressure relief valve allowing blood to flow from the left atrium to the right, reducing the load on the heart.

Examples of synthetic polymers (which may be used as a membrane component) include, but are not limited to, nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyam ides, their mixtures, blends and copolymers are suitable as a membrane material. In one embodiment, said membrane is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON®. or GORE-TEX®.), and porous or nonporous polyurethanes. In certain instances, the membrane comprises expanded fluorocarbon polymers (especially PTFE) materials described in British. Pat. No. 1,355,373; 1,506,432; or 1,506,432 or in U.S. Pat. Nos. 3,953,566; 4,187,390; or 5,276,276, the entirety of which are incorporated by reference. Included in the class of preferred fluoropolymers are polytetrafluoroethylene (PTFE), fluorinated ethylene propylene (FEP), copolymers of tetrafluoroethylene (TFE) and perfluoro(propyl vinyl ether) (PFA), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinyfluoride (PVF). Especially preferred, because of its widespread use in vascular prostheses, is ePTFE. In certain instances, the membrane comprises a combination of said materials listed above. In certain instances, the membrane is substantially impermeable to bodily fluids. Said substantially impermeable membrane can be made from materials that are substantially impermeable to bodily fluids or can be constructed from permeable materials treated or manufactured to be substantially impermeable to bodily fluids (e.g. by layering different types of materials described above or known in the art).

Additional examples of membrane materials include, but are not limited to, vinylidinefluoride/hexafluoropropylene hexafluoropropylene (HFP), tetrafluoroethylene (TFE), vinylidenefluoride, 1-hydropentafluoropropylene, perfluoro(methyl vinyl ether), chlorotrifluoroethylene (CTFE), pentafluoropropene, trifluoroethylene, hexafluoroacetone, hexafluoroisobutylene, fluorinated poly(ethylene-co-propylene (FPEP), poly(hexafluoropropene) (PHFP), poly(chlorotrifluoroethylene) (PCTFE), poly(vinylidene fluoride (PVDF), poly(vinylidene fluoride-co-tetrafluoroethylene) (PVDF-TFE), poly(vinylidene fluoride-co-hexafluoropropene) (PVDF-HFP), poly(tetrafluoroethylene-co-hexafluoropropene) (PTFE-HFP), poly(tetrafluoroethylene-co-vinyl alcohol) (PTFE-VAL), poly(tetrafluoroethylene-co-vinyl acetate) (PTFE-VAC), poly(tetrafluoroethylene-co-propene) (PTFEP) poly(hexafluoropropene-co-vinyl alcohol) (PHFP-VAL), poly(ethylene-co-tetrafluoroethylene) (PETFE), poly(ethylene-co-hexafluoropropene) (PEHFP), poly(vinylidene fluoride-co-chlorotrifluoroe-thylene) (PVDF-CTFE), and combinations thereof, and additional polymers and copolymers described in U.S. Publication 2004/0063805, incorporated by reference herein in its entirety for all purposes. Additional polyfluorocopolymers include tetrafluoroethylene (TFE)/perfluoroalkylvinylether (PAVE). PAVE can be perfluoromethylvinylether (PMVE), perfluoroethylvinylether (PEVE), or perfluoropropylvinylether (PPVE), as essentially described in U.S. Publication 2006/0198866 and U.S. Pat. No. 7,049,380, both of which are incorporated by reference herein for all purposes in their entireties. Other polymers and copolymers include, polylactide, polycaprolacton-glycolide, polyorthoesters, polyanhydrides; poly-am inoacids; polysaccharides; polyphosphazenes; poly(ether-ester) copolymers, e.g., PEO-PLLA, or blends thereof, polydimethyl-siolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters and any polymer and copolymers described in U.S. Publication 2004/0063805, incorporated by reference herein in its entirety.

The membrane components, as discussed herein, may be attached to the self-expanding frame components by using a coupling member that is generally a flat ribbon or tape having at least one generally flat surface. In certain instances, the tape member is made from expanded PTFE (ePTFE) coated with an adhesive. The adhesive may be a thermoplastic adhesive. In certain instances, the thermoplastic adhesive may be fluorinated ethylene propylene (FEP). More specifically, an FEP-coated side of the ePTFE may face toward and contacts an exterior surface of the self-expanding frame components and membrane component, thus attaching the self-expanding frame components to the membrane component. Materials and method of attaching frame components to the membrane is discussed in U.S. Pat. No. 6,042,602 to Martin, incorporated by reference herein for all purposes.

The frame components discussed herein can be fabricated from a variety of biocompatible materials. These materials may include 316L stainless steel, cobalt-chromium-nickel-molybdenum-iron alloy (“cobalt-chromium”), other cobalt alloys such as L605, tantalum, nickel-titanium alloys (e.g., Nitinol), or other biocompatible metals. In certain instances, as discussed in detail above, the frame corn ponents (and membrane) may be self-expanding. The prosthesis may be balloon expandable. In other instances, the frame components may be formed from a polymer (e.g., Polyether ether ketone (Peek)) and/or a bioabsorbable material (e.g., Poly Lactic-co-Glycolic Acid (PLGA), Polyglycolic Acid:Trim ethylene Carbonate (PGA-TMC)).

A variety of materials variously metallic, super elastic alloys, such as Nitinol, are suitable for use in these frame components. Primary requirements of the materials are that they be suitably springy even when fashioned into very thin sheets or small diameter wires. Various stainless steels which have been physically, chemically, and otherwise treated to produce high springiness are suitable as are other metal alloys such as cobalt chrome alloys (e.g., ELGILOY®), platinum/tungsten alloys, and especially the nickel-titanium alloys (e.g., Nitinol).

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable medical device, comprising:
a first frame component configured to conform to an anatomy of a patient;
a second frame component configured to conform to an anatomy of a patient wherein the first frame component and the second frame component are discrete and separate from one another; and
a conduit portion arranged between the first frame component and the second frame component, the conduit portion including a membrane connecting the first frame component and the second frame component.

2. The device of claim 1, wherein at least a portion of the conduit portion is radially unsupported by the first and second frame components within the conduit portion.

3. The device of claim 2, wherein the first and second frame components are configured to facilitate deployment of the conduit portion and maintaining a lumen through the conduit portion.

4. The device of claim 1, wherein the conduit portion is free of frame components.

5. The device of claim 1, wherein the first frame component includes a first set of elongate elements and the second frame component includes a second set of elongate elements, and the first set of elongate elements and the second set of elongate elements are non-contiguous with one another.

6. The device of claim 5, wherein the first set of elongate elements include a first plurality of support struts and wherein the second set of elongate elements include a second plurality of support struts, the first and second plurality of support struts forming a support structure within each of the elongate elements.

7. The device of claim 6, wherein the first set of elongate elements form a plurality of first lobes.

8. The device of claim 6, wherein the first set of elongate elements form a star shape.

9. The device of claim 5, wherein the first frame component forms a first side and the second frame component forms a second side, and wherein at least one of the first set of elongate elements are arranged within the first frame component without crossing into the second side and the second set of elongate elements are arranged within the second frame component without crossing into the first side.

10. The device of claim 9, wherein at least one of the first set of elongate elements and the second set of elongate elements extend within the conduit portion.

11. The device of claim 1, wherein the membrane extends to at least partially cover portions of one or both of the first frame component and the second frame component.

12. The device of claim 11, wherein the membrane is configured to promote tissue ingrowth to cover at least a portion of one or both of the first frame components and second frame components.

13. The device of claim 1, further comprising a first membrane film arranged on first frame component and a second membrane film arranged on the second frame component.

14. The device of claim 1, wherein the membrane separates the first frame components and second frame components by a gap of from 0 to 15 mm.

15. An implantable medical device for regulating blood pressure between a left and right atrium of a heart, the device comprising:
a conduit portion configured to span a septum of the heart and configured to allow fluid flow therethrough; and
a frame component including a first set of elongate elements arranged on a first side of the conduit portion and a second set of elongate elements arranged on a second side of the conduit portion with the first set of elongate elements and the second set of elongate elements being non-contiguous with one another.

16. The device of claim 15, wherein the frame component forms a first side including the first set of elongate elements and a second side including the second set of elongate elements, and wherein the first set of elongate elements are arranged within the first side and the conduit portion and the second set of elongate elements are arranged within the second side and the conduit portion.

17. The device of claim 15, wherein
the first set of elongate members and the second set of elongate members extend radially outward from the conduit portion to form first and second angles, and wherein the first and second angles are approximately 90° angles with respect to the conduit portion.

18. The device of claim 14, further including a sensor arranged with the conduit portion or the frame component and configured to sense at least one of physiologic properties, hemodynamics, biomarkers, sound, pressure, and electrolytes.

19. The device of claim 14, further comprising at least one of a coating of heparin to facilitate thromboresistance and patency of the device and a coating of paclitaxel to modulate tissue/cellular response.

20. A method for regulating blood pressure between a left and right atrium of a heart, the method comprising:
delivering the implantable medical device to a desired treatment location within a body of a patient, the implantable medical device comprising:
a conduit portion configured to span a septum of the heart and configured to allow fluid flow therethrough;
a frame component including a first set of elongate elements arranged on a first side of the conduit portion and a second set of elongate elements arranged on a second side of the conduit portion with the first set of elongate elements and the second set of elongate elements being non-contiguous with one another;
positioning the device such that the conduit portion spans a septum between the left and right atrium of the heart; and
deploying the first frame component and the second frame component such that the conduit portion opens a desired amount to provide a fluid flow path between the left and right atrium.

21. The method of claim 20, further comprising adjusting tension on the device to adjust a diameter of the conduit portion and a fluid flow velocity therethrough.

22. An implantable medical device, comprising:
a first frame component;
a second frame component, wherein the first frame component and the second frame component are discrete and separate from one another; and
a conduit portion arranged between the first frame component and the second frame component including a membrane connecting the first frame component and the second frame component configured to expand in response tension in the conduit portion imparted by expansion of the first and second frame components.

23. The device of claim 22, wherein the conduit portion is configured to span a septum between left and right atrium of a patient's and the conduit portion is configured to expand the septum in response tension in the conduit portion imparted by expansion of the first and second frame components.

24. The device of claim 23, wherein the conduit portion is configured to maintain an expanded diameter of the septum.

25. The device of claim 22, wherein the first frame component forms a first side and the second frame component forms a second side, and wherein at least one of the first set of elongate elements are arranged within the first frame component without crossing into the second side and the second set of elongate elements are arranged within the second frame component without crossing into the first side.

26. The device of claim 25, wherein at least one of the first set of elongate elements and the second set of elongate elements extend within the conduit portion.
